(19) United States
(12) Patent Application Publication
(10) Pub. No.: US 2012/0177741 A1
(43) Pub. Date: Jul. 12, 2012

(54) POSITIVELY-CHARGED POLY (D,L-LACTIDE-CO-GLYCOLIDE) NANOPARTICLES AND FABRICATION METHODS OF THE SAME

(75) Inventors: Peyman Moslemy, Mansfield, MA (US); Hong Wang, Waltham, MA (US); Michael Patane, Andover, MA (US)

(73) Assignee: EYEGATE PHARMACEUTICALS, INC., Waltham, MA (US)

(21) Appl. No.: 13/384,514

(22) PCT Filed: Sep. 29, 2010
(86) PCT No.: PCT/US10/50665

§ 371 Date: Mar. 16, 2012

Related U.S. Application Data
(60) Provisional application No. 61/246,831, filed on Sep. 29, 2009.

(51) Int. Cl. A61K 9/14; A61P 27/06; A61P 27/14; A61P 31/04; A61P 31/12; A61P 35/00; A61P 7/10; B05D 7/00; A61P 27/02; A61P 31/10; B82Y 40/00; B82Y 5/00

(52) U.S. Cl. 424/489; 427/2.14; 977/773; 977/906; 977/895

(57) ABSTRACT

The present technology provides compositions with positively-charged poly(d,l-lactide-co-glycolide) nanoparticles capable of releasing a bioactive substance in a body tissue for extended periods of time, as well as methods for manufacture of the same and methods for prophylactic and therapeutic treatment of a subject in need thereof.

### FIELD OF THE PRESENT TECHNOLOGY

This present technology relates to pharmaceutical compositions with positively-charged poly(d,l-lactide-co-glycolide) nanoparticles capable of releasing a bioactive substance in a body tissue for extended periods of time and method of making the same.

### BACKGROUND OF THE PRESENT TECHNOLOGY

Poly(d,l-lactide-co-glycolide) (PLGA) is a common biodegradable, biocompatible copolymer with a history of safe human usage in extended-release pharmaceuticals (e.g., somatropin recombinant sold under the trademark Nutropin Depot® manufactured by Alkermes for Genentech, goserelin sold under the trademark Zoladex® by AstraZeneca, leuprolide sold under the trademark Lupron Depot® by TAP Pharmaceuticals, triptorelin sold under the trademark Decapeptyl® SR by Ferring AG, and octreotide acetate sold under the trademark Sandostatin LAR® Depot by Novartis). The molecular weight of PLGA ranges from about 5,000 Daltons up to about 500,000 Daltons. The mechanism of drug release from PLGA appears to depend on both diffusion through the polymer matrix and degradation of the polymer. The copolymer is insoluble in water but soluble in many organic solvents such as ethyl acetate and acetone. Polymer degradation in aqueous environments occurs primarily by hydrolysis. The degradation products are the building monomers, lactic acid and glycolic acid, which are further metabolized to carbon dioxide and water. The degradation rate of PLGA and the drug release profile can be controlled by varying the molecular weight or the molar ratio of the two monomers in the polymer. The drug release profile can be also modified by incorporation of water soluble additives that act as a pore former.

The effectiveness of the prevention and treatment of disease and other medical conditions including, but not limited to, e.g., ocular conditions, are limited by drug-load, surface charge, physical stability and electrophoretic mobility of the currently available nanoparticles.

A need remains in the art for a composition and method for prevention and treatment of disease and other medical conditions including, but not limited to, e.g., ocular conditions.

### SUMMARY OF THE PRESENT TECHNOLOGY

The present technology relates to pharmaceutical compositions with positively-charged poly(d,l-lactide-co-glycolide) nanoparticles capable of releasing a bioactive substance in a body tissue for extended periods of time, as well as methods for manufacture and methods for prophylactic and therapeutic treatment of a subject having a disease or condition, e.g., an ocular disease or condition.

In one aspect, the present technology provides a nanoparticle composition comprising: a) PLGA or a derivative thereof; b) at least one quaternary ammonium cationic surfactant (QACS); c) a permanent positive surface charge, represented by a positive zeta potential; d) a particle size from at least about 10 nm to about 900 nm; and e) at least one bioactive agent. In one embodiment, the nanoparticles composition has zeta potential ranging from about +10 mV to about +100 mV. In one embodiment the nanoparticles composition is suitable for ocular administration.

In one aspect, the present technology provides a method of treating or preventing an ocular disease or condition in a subject, the method comprising administering to a subject in which such treatment or prevention is desired an amount of a nanoparticle composition of the present technology sufficient to treat or prevent the ocular disease or condition in the subject. In one embodiment, the ocular disease or condition is selected from the group consisting of: glaucoma, ocular inflammatory conditions such as keratitis, uveitis, intra-ocular inflammation, allergy and dry-eye syndrome ocular infections, ocular allergies, ocular infections (bacterial, fungal, and viral), cancerous growth, neo vessel growth originating from the cornea, retinal oedema, macular oedema, diabetic retinopathy, retinopathy of prematurity, degenerative diseases of the retina (macular degeneration, retinal dystrophies), and retinal diseases associated with glial proliferation.

In one aspect, the present technology provides a method for manufacturing the nanoparticle composition, comprising the steps of:

(a) preparing an oil phase by dissolving one or more bioactive agents, a one or more PLGA polymers, a one or more QACS, and optionally a one or more non-ionic surfactants in an organic solvent or a combination of organic solvents;

(b) preparing a water phase by dissolving one or more non-ionic polymeric stabilizers, optionally a one or more QACS, optionally a one or more non-ionic surfactants, and optionally a one or more pH modifying agents in purified water;

(c) emulsifying the oil and the water phase sonically, pneumatically, or mechanically under high-shear mixing;

(d) triggering the solvent diffusion-evaporation;

(e) solidifying the nanoparticles and encapsulating the active agent(s);

(f) separating the nanoparticles from the liquid medium by centrifugation or filtration; and

(g) removing the un-encapsulated ingredients from their surface by washing several times by purified water. In one embodiment, the organic solvent of step (a) has a normal boiling point from about 35° C. to about 85° C. In another embodiment, step (d) is conducted by a method selected from the group consisting of: blending the emulsion with excessive amount of an aqueous solution; depressurizing the headspace of emulsion below the atmospheric pressure while mixing; maintaining the headspace of emulsion at the atmospheric pressure while mixing; heating the emulsion at a temperature between about 35° C. and about 45° C.; or any combination thereof.

In another embodiment, the present technology provides a method for manufacturing the nanoparticle composition, comprising the steps of:

(a) preparing an internal water phase (dispersed phase of first emulsion) by dissolving a one or more bioactive agents, optionally a one or more QACS, optionally a one or more non-ionic surfactants, optionally a one or more non-ionic polymeric stabilizers, and optionally a one or more pH modifying agents in purified water;

(b) preparing an oil phase by dissolving a one or more PLGA polymers, a one or more QACS, optionally a one or more bioactive agents, and optionally a one or more non-ionic surfactants in an organic solvent or a combination of organic solvents;

(c) preparing an external water phase by dissolving a one or more non-ionic polymeric stabilizers, optionally a one or more QACS, optionally a one or more non-ionic surfactants, and optionally a one or more pH modifying agents in purified water;

(d) emulsifying the internal water phase with the oil phase sonically, pneumatically, or mechanically under high-shear mixing to form a first emulsion;

(e) emulsifying the first emulsion with the external water phase to form a double emulsion;

(f) triggering solvent diffusion-evaporation;

(g) solidifying the nanoparticles and encapsulating the active agent(s);

(h) separating the nanoparticles from the liquid medium by centrifugation or filtration; and

(i) removing un-encapsulated ingredients from the nanoparticle surface by washing several times by purified water. In one embodiment of the method, step (d) is conducted by a method selected from the group consisting of: blending the emulsion with excessive amount of an aqueous solution; depressurizing the headspace of emulsion below the atmospheric pressure while mixing; maintaining the headspace of emulsion at the atmospheric pressure while mixing; heating the emulsion at a temperature between about 35° C. and about 45° C.; or any combination thereof. In one embodiment of the method, the organic solvent of step (b) has a normal boiling point from about 35° C. to about 85° C.

In one embodiment, the present technology provides a method for manufacturing the nanoparticle composition, comprising the steps of:

(a) preparing an at least two primary oil phases by dissolving a one or more bioactive agents, a one or more PLGA polymers, a one or more QACS, and optionally one or more non-ionic surfactants in respective organic solvent or organic solvent mixtures;

(b) preparing a water phase by dissolving a one or more non-ionic polymeric stabilizers, optionally a one or more QACS, optionally a one or more non-ionic surfactants, and optionally a one or more pH modifying agents in purified water;

(c) emulsifying the at least two primary oil phases in water phase concomitantly or in succession, sonically, pneumatically, or mechanically under high-shear mixing;

(d) triggering solvent diffusion-evaporation by any of the following methods:
(d.1) blending the emulsion with excessive amount of an aqueous solution;
(d.2) depressurizing the headspace of emulsion below the atmospheric pressure while mixing;
(d.3) maintaining the headspace of emulsion at the atmospheric pressure while mixing;
(d.4) heating the emulsion at a temperature between about 35° C. and about 45° C.;
(d.5) a combination of any of methods d.1 through d.3 with method d.4.;

(e) solidifying the nanoparticles and encapsulating the active agent(s);

(f) separating the nanoparticles from the liquid medium by centrifugation or filtration; and

(g) removing the un-encapsulated ingredients from their surface by washing several times by purified water. In one embodiment of the method, the organic solvent of step (a) has a normal boiling point from about 35° C. to about 85° C.

In one embodiment, the present technology provides a method for manufacturing the nanoparticle composition, comprising the steps of:

(a) preparing an at least two internal water phases by dissolving a one or more bioactive agents, optionally a one or more QACS, optionally a one or more non-ionic surfactants, optionally a one or more non-ionic polymeric stabilizers, and optionally a one or more pH modifying agents in respective portions of purified water;

(b) preparing an at least two primary oil phases by dissolving a one or more PLGA polymers, a one or more QACS, optionally a one or more bioactive agents, and optionally a one or more non-ionic surfactants in respective organic solvent or organic solvent mixtures;

(c) preparing an external water phase by dissolving a one or more non-ionic polymeric stabilizers, optionally a one or more QACS, optionally a one or more non-ionic surfactants, and optionally a one or more pH modifying agents in purified water;

(d) emulsifying the at least two internal water phases, respectively, with the at least two oil phases sonically, pneumatically, or mechanically under high-shear mixing to establish an at least two first emulsions;

(e) emulsifying the at least two first emulsions with the external water phase, concomitantly or in succession, to form the double emulsion;

(f) triggering solvent diffusion-evaporation by any of the following methods:
(f.1) blending the emulsion with excessive amount of an aqueous solution;
(f.2) depressurizing the headspace of emulsion below the atmospheric pressure while mixing;
(f.3) maintaining the headspace of emulsion at the atmospheric pressure while mixing;
(f.4) heating the emulsion at a temperature between about 35° C. and about 45° C.;
(f.5) combination of any of methods f.1 through f.3 with method f.4;

(g) solidifying the nanoparticles and encapsulating the active agent(s);

(h) separating the nanoparticles from the liquid medium by centrifugation or filtration; and

(i) removing the un-encapsulated ingredients from their surface by washing several times by purified water. In one embodiment of the method, the organic solvent of step (b) has a normal boiling point from about 35° C. to about 85° C.

### BRIEF DESCRIPTION OF THE DRAWINGS

FIG. 1 shows the release profile of methazolamide-loaded PLGA nanoparticles. In vitro release of methazolamide from methazolamide-loaded PLGA nanoparticles. Data are represented as Mean±SD (n=4).

### DETAILED DESCRIPTION

### I. PLGA Nanoparticle Compositions of the Present Technology

In one aspect, the present technology provides drug-loaded positively-charged PLGA nanoparticle compositions. A PLGA nanoparticle composition of the present technology comprises:

a) PLGA or derivative thereof;

b) at least one quaternary ammonium surfactant (QACS);

c) a permanent positive surface charge;

d) a particle size from at least about 10 nm to about 900 nm; and

e) at least one bioactive agent.

In one embodiment, the PLGA nanoparticles are spherical in shape.

In other embodiments, the compositions of this present technology may also include other agents, for example, but not limited to, buffering agents, osmotic agents, penetration or absorption enhancers, chelants, antioxidants, preservatives, pH adjusting agents, viscosity modifying agents, lubricating agents, cryopreservative agents, and surface modifiers. These agents can be included in the formulation of nanoparticles before or during their fabrication or be added to the nanoparticle suspension after fabrication.

In another aspect the present technology provides PLGA nanoparticles that are formulated with a pharmaceutically acceptable excipient.

As used herein, the term “excipient” refers to a neutral or charged substance used as a carrier for the active agent. An excipient is typically biologically inert or nearly so.

Advantages of compositions of the present technology include, but are not limited to: (1) the use of various QACS (described below) in the formulation of nanoparticles to create highly drug-loaded, highly positively-charged, size-controlled PLGA nanoparticles; (2) the incorporation of QACS in the oil phase of emulsion to create permanently charged nanoparticles. The charge of nanoparticles cannot be compromised by multiple washing steps or variation of pH within the biologically acceptable limits; (3) the employment of a non-ionic polymeric stabilizer such as polyvinyl alcohol in the external aqueous phase in combination with QACS in the internal oil phase to produce fine nanoparticles with narrow size distribution; (4) reduced or minimized active agent partitioning into the outer aqueous phase during emulsification, and thereby, high drug loading; (5) elevated surface charge of nanoparticles, and thereby, improved physical stability and enhanced electrophoretic mobility.

The PLGA nanoparticles can be blended with other pharmaceutically acceptable active or inactive ingredients, dried by conventional processes such as spray drying or freeze drying, then packaged, and preserved under controlled storage conditions for future applications.

In one embodiment, a plurality of PLGA nanoparticles with different compositions, and thereby different extended release profiles, can be blended in order to achieve a therapeutically relevant release profile. For instance, a population of fast-releasing drug-loaded PLGA nanoparticles can be mixed with a population of slow-releasing drug-loaded nanoparticles so that a rapid onset of action followed by a sustained therapeutic action can be achieved over a certain treatment period.

A. PLGA and Derivatives Thereof Useful in the Compositions of the Present Technology

Poly(d,l-lactide-co-glycolide) (PLGA) is a common biodegradable, biocompatible copolymer with a history of safe human usage in extended-release pharmaceuticals (e.g., somatropin recombinant sold under the trademark Nutropin Depot® manufactured by Alkermes for Genentech, goserelin sold under the trademark Zoladex® by AstraZeneca, leuprolide sold under the trademark Lupron Depot® by TAP Pharmaceuticals, triptorelin sold under the trademark Decapeptyl® SR by Ferring AG, and octreotide acetate sold under the trademark Sandostatin LAR® Depot by Novartis). The molecular weight of PLGA ranges from about 5,000 Daltons up to about 500,000 Daltons. The mechanism of drug release from PLGA appears to depend on both diffusion through the polymer matrix and degradation of the polymer. The copolymer is insoluble in water but soluble in many organic solvents such as ethyl acetate and acetone. Polymer degradation in aqueous environments occurs primarily by hydrolysis. The degradation products are the building monomers, lactic acid and glycolic acid, which are further metabolized to carbon dioxide and water. The degradation rate of PLGA and the drug release profile can be controlled by varying the molecular weight or the molar ratio of the two monomers in the polymer. The drug release profile can be also modified by incorporation of water soluble additives that act as a pore former.

The rate and extent of release of a bioactive substance is influenced by: (1) the composition of nanoparticles, including but not limited to, e.g., the polymer type—molecular weight—and concentration, drug:polymer ratio, type and concentration of osmotic agents—solubility enhancers—and pore formers; (2) size distribution of nanoparticles; and (3) hydrodynamics, chemical composition, and pH of biological release environment. The release is controlled by molecular diffusion and degradation of polymer, and may take place in more than one distinguished phases with regard to the rate and extent of release. For instance, nanoparticles may exhibit an initial rapid release of their bioactive content, commonly referred to as “burst effect”, followed by a period of steady but slow release, and ending by a period of fast release due to degradation and collapse of the polymer.

In general, the rate of dissolution and release of bioactive substances may be increased in the presence of water soluble inactive ingredients such as osmotic agents, cationic surfactants, and nonionic surfactants which may also function as pore formers within the nanoparticle core. The concentration of pore forming ingredients may vary from about 0.1% to about 10% of the weight of dry composition.

The invention is related to PLGA copolymers and their derivatives such as PEGylated PLGA copolymers, PLA (polylactic acid) polymers, and PEGylated PLA (polylactic acid) diblock and triblock copolymers.

B. Charge Characteristics of Compositions of the Present Technology

The nanoparticle compositions of the present technology are characterized by their high positive charge (zeta potential >+45 mV) which in turn may contribute to their following attributes: (1) physical stability in a suspension form; (2) increased uptake by epithelial cell layers of ocular surface tissues (negatively charged at physiological pH) through adsorptive-type endocytosis in topical applications; and (3) enhanced electrophoretic mobility in electrophoretic applications.

The surface charge of nanoparticles is influenced by the composition of nanoparticles and can be varied mainly by varying the PLGA polymer(s) type (acid or ester end-group) and concentration, cationic surfactant(s) type and concentration, buffering agent(s) type and concentration, and surface modifier(s) type and concentration. In general, the surface charge of nanoparticles is also influenced by the chemistry (pH and ionic strength) of their surrounding environment.

In general, the surface charge of colloidal particles is represented by zeta potential. Zeta potential can be used to predict the electrophoretic mobility and physical stability of charged nanoparticles in a suspension in different aqueous environments. In order to prevent aggregation and successive size growth of nanoparticles, it is useful to confer repulsive forces to the particles. One of the means to confer repulsive forces to a colloidal system is by electrostatic or charge stabilization. Electrostatic or charge stabilization has the advantage of stabilizing a nanoparticle suspension by simply altering the concentration of ions surrounding the nanoparticles.

The most important mechanism to modify the surface charge of nanoparticles is by ionization of surface groups or the adsorption of charged ions. In the pharmaceutical compositions of the present technology, the positive surface charge is created by incorporating one or a combination of quaternary ammonium surfactants in the nanoparticle core formulation.

The interaction of colloidal particles in polar liquids such as water is not governed by the electrical potential at the surface of the particle, but by the effective potential of the particle and its associated ions. To utilize electrostatic control of dispersions, it is the zeta potential of the nanoparticle that must be measured rather than its surface charge. Charged particles will attract ions of opposite charge in the dispersant. Ions close to the surface are strongly bound; those further away form a more diffuse region. Within this region is a notional boundary, known as the slipping plane, within which the particle and ions act as a single entity. The potential at the slipping plane is known as the zeta potential. It has long been recognized that the zeta potential is a very good index of the magnitude of the interaction between colloidal particles and their electrophoretic mobility. Measurements of zeta potential are commonly used to assess the stability of colloidal systems. The zeta potential measured in a particular system is dependent on the chemistry of the surface, and also of the way it interacts with its surrounding environment. Therefore zeta potential must always be studied in a well defined environment (i.e. known pH and ionic strength).

An important consequence of the existence of electrical charges on the surface of particles is that they interact with an applied electric field. These effects are collectively defined as electrokinetic effects. If the motion is induced in a particle suspended in a liquid under the influence of an applied electric field, it is more specifically named electrophoresis. When an electric field is applied across an electrolyte, charged particles suspended in the electrolyte are attracted towards the electrode of opposite charge. Viscous forces acting on the particles tend to oppose this movement. When equilibrium is reached between these two opposing forces, the particles move with constant velocity. The velocity is dependent on the strength of electric field or voltage gradient, the dielectric constant of the medium, the viscosity of the medium and the zeta potential. The velocity of a particle in a unit electric field is referred to as its electrophoretic mobility. Zeta potential is related to the electrophoretic mobility by the Henry's equation:

$${U}_{e} = \frac{2 {ɛɛ}_{0} ϛ}{3 η} f ( κ a ) \qquad ( I )$$

where Uₑ is electrophoretic mobility, c is the dielectric constant of the dispersion medium, ∈₀ is the permittivity of free space, ζ is the zeta potential, η is the dynamic viscosity of the dispersion medium, and f(κa) is Henry's function. In aqueous media and moderate electrolyte concentration f(κa) is 1.5, and this is referred to as the Smoluchowski approximation. Therefore calculation of zeta potential from the electrophoretic mobility is straightforward for systems that fit the Smoluchowski model, i.e. particles larger than about 200 nm dispersed in electrolytes containing more than 1 mM salt. For smaller particles in low dielectric constant media (e.g. non-aqueous media), f(κa) becomes 1.0 and allows an equally simple calculation. This is referred to as the Huckel approximation.

C. QACS Useful in the Compositions of the Present Technology

The nanoparticles of this present technology may be formulated into pharmaceutical compositions with various hydrophilic or hydrophobic active ingredients for a large number of pharmaceutical applications. A QACS is a salt of a nitrogenous cation in which a central nitrogen atom is bonded to four organic radicals and an anion (X), of general formula R₄N⁺X⁻ which exhibits surface active properties. In a QACS generally at least one of the R groups is a long-chain (greater than 6 carbon atoms) alkyl or aryl group. Representative quaternary ammonium surfactants include, but are not limited to, those of the alkylammonium, benzalkonium, and pyridinium families. More specifically, the QACS are selected from alkyltrimethylammonium salts, alkyldimethylammonium salts, alkylmethylammonium salts, alkyldimethylbenzylammonium salts, alkylpyridinium, and alkylimidazolium salts. An exemplary list of alkylammonium surfactants is shown in Table 1.

TABLE 1
Quaternary alkylammonium surfactants

| | | Com- | | | |
|---|---|---|---|---|---|
| | | pendial | | | |
| Compound | Structure | Name | Linear Formula | MW | CAS # |
| Decyltrimethylammonium bromide | 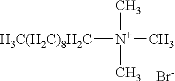 | DTAB | CH₃(CH₂)₉N(CH₃)₃(Br) | 280.29 | 2082-84-0 |
| Dodecyltrimethylammonium bromide, Lauryltrimethylammonium bromide | 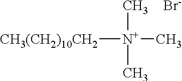 | LTAB | CH₃(CH₂)₁₁N(CH₃)₃Br | 308.34 | 1119-94-4 |
| Cetyltrimethylammonium bromide, Hexadecyltrimethylammonium bromide | 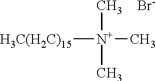 | CTAB | CH₃(CH₂)₁₅N(Br)(CH₃)₃ | 364.45 | 57-09-0 |
| Octadecyltrimethylammonium bromide | 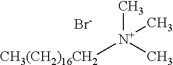 | OTAB | CH₃(CH₂)₁₇N(Br)(CH₃)₃ | 392.5 | 1120-02-1 |
| Didodecyldimethylammonium bromide | 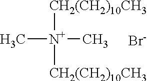 | DMAB | [CH₃(CH₂)₁₁]₂N(CH₃)₂(Br) | 462.63 | 3282-73-3 |
| Ditetradecyldimethylammonium bromide | 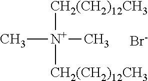 | TMAB | [CH₃(CH₂)₁₃]₂N(Br)(CH₃)₂ | 518.74 | 68105-02-2 |
| Dioctadecyldimethylammonium chloride | 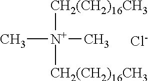 | OMAC | [CH₃(CH₂)₁₇]₂N(Cl)(CH₃)₂ | 586.5 | 107-64-2 |
| Dioctadecyldimethylammomium bromide, Distearyldimethylammonium bromide | 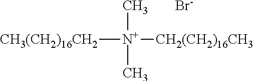 | DDAB | [CH₃(CH₂)₁₇]₂N(Br)(CH₃)₂ | 630.95 | 3700-67-2 |
| Trioctadecylmethylammonium bromide | 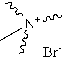 | OMAB | [CH₃(CH₂)₁₇]₃N(Br)CH₃ | 869.4 | 18262-86-7 |

In select embodiments of the present technology, the QACS is selected from the group consisting of alkyltrimethylammonium halide, alkyldimethylammonium halide, alkylmethylammonium halide, alkylethyldimethylammonium halide, alkyldimethylbenzylammonium halide, alkylpyridinium halide, and alkylimidazolium halide.

In other embodiments of the present technology the QACS is selected from decyltrimethylammonium halide, lauryltrimethylammonium halide, cetyltrimethylammonium halide, cetylethyldimethylammonium halide, octadecyltrimethylammonium halide, didodecyldimethylammonium halide, ditetradecyldimethylammonium halide, dioctadecyldimethylammonium halide, trioctadecylmethylammonium halide, or a mixture of two or more thereof.

D. Bioactive Agents Useful in the Compositions of the Present Technology

A bioactive agent is a synthetic or a natural compound which demonstrates a biological effect when introduced into a living creature. Such agents may include diagnostic and therapeutic agents including both large and small molecules intended for the treatment of acute or chronic conditions.

In some embodiments of the compositions of the present technology, therapeutic compounds include ophthalmic drugs including, but not limited to, e.g., small molecules, and biologics such as peptides, oligopeptides, proteins and antibodies, and oligonucleotides. Exemplary molecules belong to such therapeutics classes as antibacterials, antifungals, antivirals, antiglaucomatous agents, anti-histamines, anti-inflammatory agents, anti-VEGF (vascular endothelial growth factor) agents, anti-cancerous agents, decongestants, anti-diabetic agents, immunomodulators, and drugs for central nervous and movement disorders.

In one embodiment of the present technology, the bioactive agent has an aqueous solubility of greater than 1000 mg/mL (very soluble).

In one embodiment of the present technology, the bioactive agent has an aqueous solubility of 100 to 1000 mg/mL (freely soluble).

In one embodiment of the present technology, the bioactive agent has an aqueous solubility of 33 to 100 mg/mL (soluble).

In one embodiment of the present technology, the bioactive agent has an aqueous solubility of 10 to 33 mg/mL (sparingly soluble).

In one embodiment of the present technology, the bioactive agent has an aqueous solubility of 1 to 10 mg/mL (slightly soluble).

In one embodiment of the present technology, the bioactive agent has an aqueous solubility of 0.1 to 1 mg/mL (very slightly soluble).

In one embodiment of the present technology, the bioactive agent has an aqueous solubility of less than 0.1 mg/mL (practically insoluble).

In one embodiment of the present technology, the bioactive agent comprises between 1% and 90% of the nanoparticle mass, preferably between 10% and 70%, and more preferably between 20% and 50% of the nanoparticle mass.

E. Particle Size of the Compositions of the Present Technology

In one embodiment, the PLGA nanoparticles of the present technology have a diameter from about 10 nm to about 900 nm.

In one embodiment, the PLGA nanoparticles of the present technology have a diameter from about 50 nm to about 700 nm.

In one embodiment, the PLGA nanoparticles of the present technology have a diameter from about 100 nm to about 500 nm.

In one embodiment, the PLGA nanoparticles of the present technology have a diameter from about 150 nm to about 300 nm.

F. Other Agents Useful in the Compositions of the Present Technology

### Buffering Agents

In one embodiment of the invention, the compositions of the present technology optionally comprise at least one buffering agent. Such buffering agent may be used to control the pH of formulation that otherwise may change as a result of chemical or electrochemical interactions during use or storage of the formulation.

In some embodiments, the buffer agent(s) comprise an amino acid or a combination of amino acids with cationic behavior. In another embodiment, mixtures of a cationic amino acid buffer and an anionic acid buffer may also be used. Cationic amino acids useful in the compositions/formulations of the present technology include, but are not limited to, e.g., arginine, aspartic acid, cycteine, glutamic acid, histidine, lysine, and tyrosine. Anionic acids useful in the compositions/formulations of the present technology include, but are not limited to, e.g., acetic acid, adipic acid, aspartic acid, benzoic acid, citric acid, ethylenediamine tetracetic acid, formic acid, fumaric acid, glutamic acid, glutaric acid, maleic acid, malic acid, malonic acid, phosphoric acid, and succinic acid.

In select embodiments, the buffering agent comprises an amino acid or a combination of amino acids with anionic behavior. In other embodiments, mixtures of an anionic amino acid buffer and an anionic acid buffer and a cationic base or cationic amino acid buffer may also be used. Anionic amino acids useful in the compositions/formulations of the present technology include, but are not limited to, e.g., cysteine, histidine, and tyrosine. Anionic acid buffers useful in the compositions/formulations of the present technology include, but are not limited to, e.g., acetic acid, adipic acid, benzoic acid, carbonic acid, citric acid, ethylenediamine tetracetic acid, fumaric acid, glutamic acid, lactic acid, maleic acid, malic acid, malonic acid, phosphoric acid, tartaric acid, and succinic acid. Cationic bases and amino acids useful in the compositions/formulations of the present technology include, but are not limited to, e.g., arginine, histidine, imidazole, lysine, triethanolamine, and tromethamine.

In some embodiments, buffering agents include zwitterions. Zwitterions useful in the compositions/formulations of the present technology include, but are not limited to, e.g., N-2(2-acetamido)-2-aminoethane sulfonic acid (ACES), N-2-acetamido iminodiacetic acid (ADA), N,N-bis(2-hydroxyethyl)-2-aminoethane sulfonic acid (BES), 2-[Bis-(2-hydroxyethyl)-amino]-2-hydroxymethyl-propane-1,3-diol (Bis-Tris), 3-cyclohexylamino-1-propane sulfonic acid (CAPS), 2-cyclohexylamino-1-ethane sulfonic acid (CHES), N,N-bis(2-hydroxyethyl)-3-amino-2-hydroxypropane sulfonic acid (DIPSO), 4-(2-hydroxyethyl)-1-piperazine propane sulfonic acid (EPPS), N-2-hydroxyethylpiperazine-N′-2-ethane sulfonic acid (HEPES), 2-(N-morpholino)-ethane sulfonic acid (MES), 4-(N-morpholino)-butane sulfonic acid (MOBS), 2-(N-morpholino)-propane sulfonic acid (MOPS), 3-morpholino-2-hydroxypropanesulfonic acid (MOPSO), 1,4-piperazine-bis-(ethane sulfonic acid) (PIPES), piperazine-N,N′-bis(2-hydroxypropane sulfonic acid) (POPSO), N-tris(hydroxymethyl)methyl-2-aminopropane sulfonic acid (TAPS), N-[tris(hydroxymethyl)methyl]-3-amino-2-hydroxypropane sulfonic acid (TAPSO), N-tris(hydroxymethyl) methyl-2-aminoethane sulfonic acid (TES), and 2-Amino-2-hydroxymethyl-propane-1,3-diol (Tris).

In select embodiments, buffering agents include a polymer or a combination of polymers with anionic or cationic behavior. The polymeric buffer may be any polymer which ionizes at a given pH by consuming hydrogen ions or hydroxyl ions and maintains the pH of the nanoparticle composition within a desired range. Anionic polymer useful in the compositions/formulations of the present technology include, but are not limited to, e.g., poly(acrylic acid), poly(acrylic acid) crosslinked with polyalkenyl ethers or divinyl glycol, poly(methacrylic acid), styrene/maleic anhydride copolymers, methyl vinyl ether/maleic anhydride copolymers, poly(vinyl acetate phthalate), cellulose acetate phthalate, cellulose acetate trimellitate, hydroxypropyl methylcellulose phthalate, hydroxypropyl methylcellulose acetate succinate, ethyl acrylate/methacrylic acid copolymers, methyl methacrylate/methacrylic acid copolymers, and alginic acid. Cationic polymer useful in the compositions/formulations of the present technology include, but are not limited to, e.g., polyvinylpyridine, methyl methacrylate/butyl methacrylate/dimethylaminoethyl methacrylate terpolymers, vinylpyrrolidone/quaternized dimethyl aminoethyl methacrylate copolymers, vinylcaprolactam/vinylpyrrolidone/dimethyl aminoethyl methacrylate terpolymers, and chitosan.

In other embodiments, the buffer composition is a crosslinked polymer or a combination of polymers with anionic or cationic behavior. In one embodiment, the polymeric buffer is an ion exchange resin. Ion exchange resins useful in the compositions/formulations of the present technology include, but are not limited to, e.g., methacrylic acid/divinylbenzene copolymers and styrene/divinylbenzene copolymers. Methacrylic acid/divinylbenzene copolymers have weak acid (carboxyl group) functionality and are available in hydrogen or potassium form. Styrene/divinylbenzene polymers have either strong acid (sulfonate group) or strong base (tertiary amine group) functionality. The former resins are available in hydrogen, sodium or calcium form and while the latter resins are available in chloride form.

In other embodiments, the buffer composition is a crosslinked polymer or a combination of polymers with zwitterionic behavior. Zwitterionic polymers useful in the compositions/formulations of the present technology include, but are not limited to, e.g., poly(2-acrylamido-2-methyl-1-propane sulfonic acid) hydrogels (generally referred to as PolyAMPS), PolyAMPS/hyaluronic acid interpenetrating polymer network (IPN) hydrogels, cross-linked copolymers of AMPS and 2-hydroxyethyl methacrylate (HEMA), cross-linked copolymers of AMPS and 2-dimethylamino ethyl methacrylate (DMAEMA), and cross-linked copolymers of AMPS and acrylic acid.

In other embodiments, buffering agents useful in the compositions/formulations of the present technology include, but are not limited to, e.g., phosphate, citrate, or acetate buffers or combinations thereof.

### Osmotic Agents

In some embodiments, the formulations of the present technology optionally contain at least one osmotic agent (or tonicity adjusting agent) sufficient to render the composition acceptable for administration to a human or an animal. Exemplary osmotic agents are sodium chloride, sodium borate, sodium acetate, sodium phosphates, sodium sulfate, potassium sulfate, calcium sulfate, magnesium sulfate, sodium hydroxide, and hydrochloric acid, mannitol, sorbitol, glucose, sucrose, lactulose, trehalose, and glycerol. Polyols, such as erythritol components, xylitol components, inositol components, and the like and mixtures thereof, are effective tonicity/osmotic agents, and may be included, alone or in combination with glycerol and/or other compatible solute agents, in the invention compositions. Other non-ionic tonicity adjusting agents include polyethylene glycols (PEG), polypropylene glycols (PPG) and mixtures thereof.

### Penetration Enhancers

Compositions/formulations of the present technology optionally include one or more agents to enhance the body tissue penetration or absorption of nanoparticles. For instance, the epithelium is the main barrier to drug penetration through the cornea. It is possible to enhance the penetration of drugs through the epithelium by promoting drug partition into the epithelium, thereby enhancing the overall absorption of drugs applied to the eye. The penetration enhancer generally acts to make the cell membranes less rigid and therefore more amenable to allowing passage of drug molecules between cells. The penetration enhancers preferably exert their penetration enhancing effect immediately upon application to the eye and maintain this effect for a period of approximately five to ten minutes. The penetration enhancers are required to be pharmacologically inert and chemically stable, to have a high degree of potency in terms of both specific activity and reversible effects on cornea permeability, and to be both nonirritating and nonsensitizing. The penetration enhancers and any metabolites thereof must also be non-toxic to ophthalmic tissues.

Penetration enhancers useful in the compositions/formulations of the present technology include, but are not limited to, e.g., surfactants (including bile acids including deoxycholic acid, taurocholic acid, taurodeoxycholic acid, and the like; bile salts such as sodium cholate and sodium glycocholate); fatty acids such as capric acid; preservatives such as benzalkonium chloride, chlorhexidine digluconate, parabens such as methylparaben and propylparaben, chlorobuthanol, and so on; chelating agents such as ethylenediamine tetraacetic acid (EDTA) and its sodium salts; polyoxyethylene sorbitan fatty acid esters such as polyoxyethylene sorbitan monolaurate (polysorbate 20, Tween® 20); polyoxyethylene lauryl ethers such as polyoxyethylene (23) lauryl ether (Brij 35); and other compounds such as dimethyl sulfoxide (DMSO), 1-dodecylazayl-cycloheptan-2-one (Azone®), hexamethylene lauramide, decylmethylsulfoxide, decamethonium bromide, saponin, and sodium fusidate. A complete list of the above penetration enhancers is provided by Sasaki et al. in: Critical Reviews in Therapeutic Drug Carrier Systems, 16(1):85-146 (1999).

Other penetration enhancers useful in the compositions/formulations of the present technology include, but are not limited to, e.g., saccharide surfactants, such as dodecylmaltoside (DDM) and monoacyl phosphoglycerides such as lysophosphatidylcholine. The saccharide surfactants and monoacyl phosphoglycerides which may be utilized as penetration enhancers in the present invention are known compounds. The use of such compounds to enhance the penetration of ophthalmic drugs is described in the U.S. Pat. No. 5,221,696 and the U.S. Pat. No. 5,369,095, respectively.

### Chelants

In another embodiment, the compositions of the present technology may contain at least one chelating agent selected from the group consisting of sodium citrate and EDTA and its sodium salts. A chelant, as used herein, chelates metal ions which may catalyze the degradation of the encapsulated drug.

### Antioxidants

In one embodiment, the compositions/formulations of the present technology may contain at least one antioxidant. Antioxidants useful in the compositions/formulations of the present technology include, but are not limited to, e.g., alpha tocopherol (Vitamin E); cysteine; taurine; citric acid, ascorbic acid, ascorbyl palmitate, EDTA and its sodium salts; sodium bisulfite, and sodium metabisulfite. An antioxidant, as used herein, prevents or reduces the degradation of a drug which could otherwise degrade through oxidative pathways.

### Preservatives

In one embodiment of the invention, the compositions/formulations may contain at least one preservative. A preservative, as used herein, is an additive which inhibits microbial growth and or kills microorganisms which inadvertently contaminate a pharmaceutical composition upon exposure to the surroundings. The preservative may be selected from a variety of well known preservatives, including hydrophobic or non-charged preservatives, anionic preservatives, and cationic preservatives. A preservative enhancing agent, as used herein, refers to an additive which increases the preservative effectiveness of a preservative, or the preservative effectiveness of a preserved formulation, but which would not typically be used solely to preserve a pharmaceutical composition.

Cationic preservatives useful in the compositions/formulations of the present technology include, but are not limited to, e.g., polymyxin B sulfate, quaternary ammonium compounds, poly(quaternary ammonium) compounds, p-hydroxybenzoic acid esters, benzalkonium chloride, benzoxonium chloride, cetylpridinium chloride, benzethonium chloride, cetyltrimethyl ammonium bromide, chlorhexidine, poly(hexamethylene biguanide), and mixtures thereof.

Anionic preservatives useful in the compositions/formulations of the present technology include, but are not limited to, e.g., sorbic acid; 1-octane sulfonic acid (monosodium salt); 9-octadecenoic acid (sulfonated); ciprofloxacin; dodecyl diphenyloxide-disulfonic acid; ammonium, potassium, or sodium salts of dodecyl benzene sulfonic acid; sodium salts of fatty acids or tall oil; naphthalene sulfonic acid; sodium salts of sulfonated oleic acid; organic mercurials such as thimerosal (sodium ethylmercurithiosalicylate); thimerfonate sodium (sodium p-ethylmercurithiophenylsulfonate).

Hydrophobic or non-ionic preservatives useful in the compositions/formulations of the present technology include, but are not limited to, e.g., without limitation thereto, 2,3-dichloro-1,4-naphthoquinone; 3-methyl-4-chlorophenol; 8-hydroxyquinoline and derivatives thereof; benzyl alcohol; phenethyl alcohol; bis(hydroxyphenyl) alkanes; bisphenols; chlorobutanol; chloroxylenol; dichlorophen[2,2′-methylene-bis(4-chlorophenol)]; ortho-alkyl derivatives of para-bromophenol and para-chlorophenol; oxyquinoline; para-alkyl derivatives of ortho-chlorophenol and ortho-bromophenol; pentachlorophenyl laurate; phenolic derivatives such as 2-phenylphenol, 2-benzyl-4-chlorophenol, 2-cyclopentyl-4-chlorophenol, 4-t-amylphenol, 4-t-butylphenol, and 4- and 6-chloro-2-pentylphenol; phenoxy fatty acid polyester; phenoxyethanol; methylparaben, propylparaben, and butylparaben.

### pH Adjusting Agents

In one embodiment, the compositions/formulations of the present technology may contain at least one pH adjusting agent. pH adjusting agents useful in the compositions/formulations of the present technology include, but are not limited to, e.g., hydrochloric acid, citric acid, phosphoric acid, acetic acid, tartaric acid, sodium hydroxide, potassium hydroxide, sodium carbonate and sodium bicarbonate.

### Viscosity Modifying Agents

In one embodiment, the compositions/formulations of the invention may contain at least one viscosity modifying agent. Viscosity modifying agents useful in the compositions/formulations of the present technology include, but are not limited to, e.g., cellulose derivatives such as hydroxymethylcellulose, hydroxyethylcellulose, hydroxypropylcellulose, hydroxypropylmethylcellulose, methylcellulose, and carboxymethylcellulose; poly(N-vinylpyrrolidone); poly(vinylalcohol); polyethylene oxides; polyoxyethylene-polyoxypropylene copolymers (poloxamers); polysaccharides such as alginates; carrageenans; guar gum, karaya gum, gellan gum, agarose, locust bean gum, tragacanth gum, xanthan gum, and chitosan; hyaluronic acid; lecithin; and carbomer polymers (Carbopol®).

### Lubricating Agents

In one embodiment, the compositions/formulations of the present technology may contain at least one lubricating agent. Lubricating agents useful in the compositions/formulations of the present technology include, but are not limited to, e.g., cellulose derivatives such as hydroxymethylcellulose, hydroxyethylcellulose, hydroxypropylcellulose, and hydroxypropylmethylcellulose.

### Cryopreservation Agents

In one embodiment, the compositions/formulations of the present technology may contain at least one cryopreservation agent. Cryopreservation agents useful in the compositions/formulations of the present technology include, but are not limited to, e.g., carbohydrates including saccharides—disaccharides—and sugar alcohols, glycerol, polyalkoxyethers, PEG-fatty acids and lipids, biologically-based surfactants, and other surface active agents.

Cryoprotectants used in nanoparticle suspensions are disclosed in U.S. Pat. No. 5,302,401. In the '401 patent, cryoprotectants inhibit the agglomeration of nanoparticles during the process of lyophilization. Examples of suitable cryoprotectants include carbohydrates such as sucrose, xylose, glucose, and sugar alcohols such as mannitol and sorbitol, surface active agents such as the polysorbates (Tween®s), as well as glycerol and dimethylsulfoxide. Cryoprotectants may also include water-soluble polymers such as polyvinylpyrrolidone (PVP), starch, and polyalkoxy ethers such as polyethylene glycols, polypropylene glycols, and poloxamers. Biologically derived cryoprotectants include albumin. Yet another class of cryoprotectant includes PEGylated lipids, such as Solutol® HS 15 (polyethylene glycol 660 12-hydroxystearate).

### Surface Modifiers

In one embodiment, the compositions/formulations of the present technology may contain at least one surface modifying agent. Surface modifiers useful in the compositions/formulations of the present technology include, but are not limited to, e.g., nonionic surfactants and surface active biological modifiers. Nonionic surfactant useful in the compositions/formulations of the present technology include, but are not limited to, e.g., polyoxyethylene fatty alcohol ethers, polyoxyethylene sorbitan fatty acid esters, polyoxyethylene fatty acid esters, polyoxyethylene-derivatized lipids such as mPEG-PSPC (palmitoyl-stearoyl-phophatidylcholine), mPEG-PSPE (palmitoyl-stearoyl-phophatidylethanolamine), sorbitan esters, glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxamers), polaxamines, methylcellulose, hydroxycellulose, hydroxypropylcellulose, hydroxypropyl methylcellulose, noncrystalline cellulose, polysaccharides, starch, starch derivatives, hydroxyethylstarch, polyvinyl alcohol, and polyvinylpyrrolidone.

### II. Methods of Making the PLGA Nanoparticles of the Present Technology

In one aspect, the present technology provides phase dispersion methods for making drug-loaded positively-charged PLGA nanoparticles involving at least one water (aqueous) phase and at least one oil (non-aqueous) phase. The methods of making drug-loaded positively charged PLGA nanoparticles of present technology differ from other methods known in the art in several ways. First, in some embodiments, at least one quaternary ammonium cationic surfactant (QACS) is used along with the PLGA polymer in the oil phase to confer the particle positive charge to the nanoparticles. Second, in some embodiments, at least one polymeric stabilizer such as polyvinyl alcohol or methylcellulose is present in the water phase along with the QACS in the oil phase.

In some embodiments, drug-loaded positively charged PLGA nanoparticles of the present technology are fabricated through an emulsification-solvent diffusion-evaporation process. Oil-in-water (O/W) emulsion, or water-in-oil-in-water (W₁/O/W₂) double emulsion, or emulsion combined with phase separation including oil-in-oil-in-water (O₁/O₂/W), or water-in-oil-in-oil (W/O₁/O₂) systems are useful in the methods of making the drug-loaded positively-charged PLGA nanoparticles of the present technology.

In a preferred embodiment of the present technology, the drug-loaded positively-charged PLGA nanoparticles are fabricated through O/W emulsification-solvent diffusion-evaporation. The oil phase (internal phase or dispersed phase) is prepared by dissolving one or more bioactive agents, one or more PLGA polymers, one or more QACS, and optionally one or more non-ionic surfactants in an organic solvent or a combination of organic solvents. The preferred organic solvents have a normal boiling point from about 35° C. to about 85° C. The water phase (external phase or continuous phase) is prepared by dissolving one or more non-ionic polymeric stabilizers, optionally one or more QACS, optionally one or more non-ionic surfactants, and optionally one or more pH modifying agents in purified water. The oil phase is emulsified in the water phase sonically, pneumatically, or mechanically under high-shear mixing. Once the emulsion is established, solvent diffusion-evaporation is triggered by blending the emulsion with excessive amount of an aqueous solution, hereinafter referred to as ‘quench medium’. The outward diffusion of solvent from oil globules in the emulsion leads to solidification of nanoparticles, and encapsulation of active agent(s). The rate of solvent diffusion, and therefore the rate of formation of nanoparticles, can be modified by adding the quench medium under controlled temperature and pressure conditions. Mildly high temperatures (35-45° C.) and sub-atmospheric pressures (0.6-0.8 bar) may accelerate the removal of solvent(s) and the formation of nanoparticles. The nanoparticles are separated from the liquid medium by centrifugation or filtration techniques, and then washed several times by purified water to remove un-encapsulated ingredients from their surface. The conventional methods of separation and refinement of nanoparticles are known to those skilled in the art.

In another embodiment of the present technology, the drug-loaded positively-charged PLGA nanoparticles are fabricated through W₁/O/W₂ emulsification-solvent diffusion-evaporation. The internal water phase (dispersed phase of first emulsion) is prepared by dissolving one or more bioactive agents, optionally one or more QACS, optionally one or more non-ionic surfactants, optionally one or more non-ionic polymeric stabilizers, and optionally one or more pH modifying agents in purified water. The oil phase (continuous phase of first emulsion) is prepared by dissolving one or more PLGA polymers, one or more QACS, optionally one or more bioactive agents, and optionally one or more non-ionic surfactants in an organic solvent or a combination of organic solvents. The preferred organic solvents have a normal boiling point from about 35° C. to about 85° C. The external water phase (continuous phase of second emulsion) is prepared by dissolving one or more non-ionic polymeric stabilizers, optionally one or more QACS, optionally one or more non-ionic surfactants, and optionally one or more pH modifying agents in purified water. The internal water phase is emulsified in the oil phase sonically, pneumatically, or mechanically under high-shear mixing. Once the first emulsion is established, it is emulsified in the external water phase to form the double emulsion. Once the double emulsion is established, solvent diffusion-evaporation is triggered by blending the emulsion with excessive amount of a quench medium. The outward diffusion of solvent from oil globules in the emulsion leads to solidification of nanoparticles, and encapsulation of active agent(s). The rate of solvent diffusion, and therefore the rate of formation of nanoparticles, can be modified by adding the quench medium under controlled temperature and pressure conditions. Mildly high temperatures (35-45° C.) and sub-atmospheric pressures (0.6-0.8 bar) may accelerate the removal of solvent(s) and the formation of nanoparticles. The nanoparticles are separated from the liquid medium by centrifugation or filtration techniques, and then washed several times by purified water to remove un-encapsulated ingredients from their surface. The conventional methods of separation and refinement of nanoparticles are known to those skilled in the art.

The refined nanoparticles can be blended with other pharmaceutically acceptable active or inactive ingredients, dried by conventional processes such as spray drying or freeze drying, packaged, and preserved under controlled storage conditions for future applications.

In conventional emulsification-solvent evaporation methods, a QACS is usually added to the outer aqueous phase of the emulsion while the bioactive agent is added to either the organic phase or the inner aqueous phase. These methods suffer from low encapsulation efficiency of active agents. The encapsulation remains a challenge irrespective of active agent's degree of hydrophobicity because of its rapid partitioning to the external aqueous phase. The presence of QACS in the external aqueous phase promotes the dissolution of hydrophilic and hydrophobic compounds through formation of micellar structures. Addition of QACS to the oil phase in this present technology, unlike the conventional methods, enables fabrication of positively charged nanoparticles with high encapsulation efficiency. It is believed that during emulsification the QACS molecules rearrange within the surface layers of oil globules prior to particle solidification such that their hydrophilic charged ends face the external aqueous phase while their hydrophobic tails extend towards the particle core. This arrangement allows permanent entrapment of QACS within the nanoparticles.

The preferred organic solvents in this present technology include ethyl acetate, acetone, methylene chloride, and polyethylene glycol (MW 400).

In another embodiment, the present technology provides a method for manufacturing the nanoparticle composition, comprising the steps of:

(a) preparing at least two primary oil phases by dissolving one or more bioactive agents, one or more PLGA polymers, one or more QACS, and optionally one or more non-ionic surfactants in respective organic solvent or organic solvent mixtures; The preferred organic solvents have a normal boiling point from about 35° C. to about 85° C.

(b) preparing a water phase by dissolving one or more non-ionic polymeric stabilizers, optionally one or more QACS, optionally one or more non-ionic surfactants, and optionally one or more pH modifying agents in purified water;

(c) emulsifying the at least two primary oil phases in water phase concomitantly or in succession, sonically, pneumatically, or mechanically under high-shear mixing;

(d) triggering the solvent diffusion-evaporation by any of the following methods:
(d.1) blending the emulsion with excessive amount of an aqueous solution;
(d.2) depressurizing the headspace of emulsion below the atmospheric pressure while mixing;
(d.3) maintaining the headspace of emulsion at the atmospheric pressure while mixing;
(d.4) heating the emulsion at mildly high temperatures, i.e. 35-45° C.;
(d.5) combination of any of methods d.1 through d.3 with method d.4.

(e) solidifying the nanoparticles and encapsulating the active agent(s);

(f) separating the nanoparticles from the liquid medium by centrifugation or filtration; and

(g) removing the un-encapsulated ingredients from their surface by washing several times by purified water.

In another embodiment, the present technology provides a method for manufacturing the nanoparticle composition comprising the steps of:

(a) preparing at least two internal water phases (dispersed phases of first emulsion) by dissolving one or more bioactive agents, optionally one or more QACS, optionally one or more non-ionic surfactants, optionally one or more non-ionic polymeric stabilizers, and optionally one or more pH modifying agents in respective portions of purified water;

(b) preparing at least two primary oil phases (continuous phases of first emulsions) by dissolving one or more PLGA polymers, one or more QACS, optionally one or more bioactive agents, and optionally one or more non-ionic surfactants in respective organic solvent or organic solvent mixtures; The preferred organic solvents have a normal boiling point from about 35° C. to about 85° C.

(c) preparing the external water phase (continuous phase of second emulsion) by dissolving one or more non-ionic polymeric stabilizers, optionally one or more QACS, optionally one or more non-ionic surfactants, and optionally one or more pH modifying agents in purified water;

(d) emulsifying the at least two internal water phases respectively with the at least two oil phases sonically, pneumatically, or mechanically under high-shear mixing to establish the at least two first emulsions; The at least two emulsions are different from each other in at least two aspects, compositions and concentrations of active ingredients and PLGA polymers.

(e) emulsifying the at least two first emulsions with the external water phase, concomitantly or in succession, to form the double emulsion;

(f) triggering the solvent diffusion-evaporation by any of the following methods:
(f.1) blending the emulsion with excessive amount of an aqueous solution;
(f.2) depressurizing the headspace of emulsion below the atmospheric pressure while mixing;
(f.3) maintaining the headspace of emulsion at the atmospheric pressure while mixing;
(f.4) heating the emulsion at mildly high temperatures, i.e. 35-45° C.;
(f.5) combination of any of methods f.1 through f.3 with method f.4.

(g) solidifying the nanoparticles and encapsulating the active agent(s);

(h) separating the nanoparticles from the liquid medium by centrifugation or filtration; and

(i) removing the un-encapsulated ingredients from their surface by washing several times by purified water.

In one embodiment, the at least two primary oil phases or emulsions are physically and/or chemically different and are prepared using different kinds of drugs and/or PLGA polymers. In another embodiment, the at least two primary oil phases or emulsions can be prepared using one drug and one polymer. In this case, the parameters determining the difference in physical and/or chemical properties between two or more primary oil phases or emulsions include the weight ratio of drug to polymer, the weight ratio of drug or polymer to organic solvent, the weight ratio between organic solvents (if two or more organic solvents are used), and the weight ratio of an organic solvent to an aqueous solvent (if the drug is water soluble, that is, when double emulsion is used).

In regard to the dispersion and emulsification, in one embodiment the two or more primary oil phases or emulsions are added to the external water phase in parallel or in succession. The solidification of nanoparticles in the two oil phases or emulsions is then achieved concurrently by triggering the solvent diffusion-evaporation step. In another embodiment, the two or more primary oil phases or emulsions are added to the external water phase in succession. One of the primary oil phases or emulsions is first dispersed in the external water phase which is allowed to undergo a change in its physical or chemical conditions (i.e. homogenization speed or intensity, temperature, pressure, water phase amount, and the concentrations of inactive ingredients) leading to complete or partial solidification of respective nanoparticles. The other oil phases are then dispersed in the water phase in sequence and respective nanoparticles are formed by varying the physical or chemical conditions of emulsions.

### III. Methods of Using the PLGA Nanoparticles of the Present Technology

A wide variety of ocular conditions such as glaucoma, ocular inflammatory conditions such as keratitis, uveitis, intra-ocular inflammation, allergy and dry-eye syndrome ocular infections, ocular allergies, ocular infections (bacterial, fungal, and viral), cancerous growth, neo vessel growth originating from the cornea, retinal oedema, macular oedema, diabetic retinopathy, retinopathy of prematurity, degenerative diseases of the retina (macular degeneration, retinal dystrophies), and retinal diseases associated with glial proliferation may be prevented or treated using the positively-charged nanoparticle compositions according to the present technology.

### EXAMPLES

The following examples are intended to be non-limiting illustrations of certain embodiments of the present invention. All references cited are hereby incorporated herein by reference in their entireties.

### Example 1

### Fabrication of Dexamethasone-Loaded Positively-Charged PLGA Nanoparticles

Two lots of dexamethasone-loaded positively-charged PLGA (poly(d,l-lactide-co-glycolide)) nanoparticles were fabricated with water-in-oil emulsification-solvent diffusion-evaporation method. One lot (# LBN0023-076-1) was prepared with PLGA 8515 DLG 1.5CE (lot #, LX00279-45, Lakeshore Biomaterials, Birmingham, Ala.) and the other (# LBN0023-076-2) was made with PLGA 7030 (lot #578399, Polysciences, Warrington, Pa.).

Oil phase was prepared by dissolving about 200 mg PLGA, about 60 mg dexamethasone, and about 25 mg ditetradecyldimethylammonium bromide (TMAB) in a mixture of ethyl acetate (6 mL) and acetone (4 mL). Aqueous phase was prepared by dissolving about 200 mg polyvinyl alcohol (PVA) in 20 mL water (WFI, water for injection). Target compositions of oil phase and aqueous phase solutions are provided in Table 2. Both oil phase and aqueous phase solutions were filtered through 200 nm syringe filters prior to emulsification. The oil phase was emulsified in the aqueous phase by high-shear homogenization using a rotor/stator homogenizer operating at 16 k RPM. Immediately after homogenization started in PVA solution, the organic phase was added dropwise with a syringe during 2 min. The resulting emulsion was homogenized for about 10 min. Solidification of PLGA nanoparticles, and thereby encapsulation of dexamethasone in nanoparticles, was triggered by transferring the O/W emulsion into a vessel containing 200 mL WFI under stirring. Stirring was continued for 4½ hours in a fume hood at room temperature (RT) in order to remove solvents. A milky suspension of nanoparticles was obtained. The suspension was transferred into centrifuge tubes, and then centrifuged at 15 k RPM (RCF 27,200×g) for 15 minutes at 4° C. After the supernatant was removed, the pellet was washed with WFI (20 mL) by re-suspending the nanoparticles using a vortex mixer. The suspension was centrifuged again and the supernatant was removed. The pellet was washed once more with WFI (20 mL). Final pellet after removing supernatant was reconstituted in WFI (15 mL) by vortexing and stored at 4° C.

TABLE 2
Target compositions of oil phase and aqueous phase solutions

| Emulsion | | | | |
|---|---|---|---|---|
| Phase | Ingredient | Function | Quantity | |
| Oil | Dexamethasone | Active agent/Corticosteroid | 60 | mg |
| | PLGA | Carrier/Release controlling | 200 | mg |
| | | polymer | | |
| | TMAB | Surface modifier/Surfactant | 25 | mg |
| | Ethyl acetate | Solvent | 6 | mL |
| | Acetone | Solvent | 4 | mL |
| Aqueous | PVA | Emulsion stabilizer/Surface | 200 | mg |
| | | modifier | | |
| | WFI | Solvent | 20 | mL |

Particle size and zeta potential measurements were performed by means of a Zetasizer Nano ZS90 (Malvern Instruments, Westborough, Mass.) using reconstituted nanoparticles in WFI.

About 1 mL of reconstituted suspension was transferred to a pre-weighed 1.5 mL Eppendorf tube and centrifuged at 13 k RPM for 30 minutes at RT. After the supernatant was removed, the pellet was dried under vacuum overnight at room temperature. The tube containing dry pellet was weighed and the weight of dry pellet was obtained. Dry pellet was then dissolved in 0.1 mL of dimethyl sulfoxide (DMSO). An aliquot of the resulting solution was analyzed by high performance liquid chromatography (HPLC). Drug loading was calculated from the following Equation II:

$$Drug Loading = \frac{( weight of drug in dry pellet )}{( weight of dry pellet )} \times 100 \% \qquad ( II )$$

Encapsulation efficiency was calculated from the following Equation III:

$$Encapsulation Efficiency = \frac{( total weight of drug encapsulated )}{( weight of drug initially added )} \times 100 \% \qquad ( III )$$

The characterization results are given in Table 3.

TABLE 3
Characteristics of dexamethasone-loaded positively-charged PLGA
nanoparticles

Formulation Lot #

| Attributes | LBN0023-076-1 | LBN0023-076-2 |
|---|---|---|
| Drug loading | 24% | 21% |
| Encapsulation | 79% | 67% |
| Efficiency | | |
| Mean Particle Size | 162 nm | 160 nm |
| Polydispersity Index | 0.08 | 0.07 |
| Zeta potential | (+) 55 mV | (+) 48 mV |

### Example 2

### Fabrication of Dexamethasone-Loaded Positively-Charged PLGA Nanoparticles

Two lots of dexamethasone-loaded positively-charged PLGA nanoparticles were fabricated with water-in-oil emulsification-solvent diffusion-evaporation method. PLGA 8515 DLG 1.5CE (lot #, LX00279-45, Lakeshore Biomaterials, Birmingham, Ala.) was used in both formulations. Unlike the formulations of Example 1 wherein PVA was employed as emulsion stabilizer in the aqueous phase, these formulations were prepared with TMAB dissolved in the aqueous phase, serving as the emulsion stabilizer. One lot (# LBN0023-119-1) was prepared with 0.13% (w/v) TMAB and the other (# LBN0023-119-2) was made with 0.25% (w/v) TMAB. No PVA was used in the formulations.

Oil phase was prepared by dissolving about 200 mg PLGA and about 60 mg dexamethasone in a mixture of ethyl acetate (6 mL) and acetone (4 mL). Aqueous phase was prepared by dissolving TMAB in 20 mL water (WFI, water for injection). Target compositions of oil phase and aqueous phase solutions are provided in Table 4.

TABLE 4
Target compositions of oil phase and aqueous phase solutions

| | | | Quantity | | | |
|---|---|---|---|---|---|---|
| Emulsion | | | LBN0023- | | LBN0023- | |
| Phase | Ingredient | Function | 119-1 | | 119-2 | |
| Oil | Dexamethasone | Active agent/ | 60 | mg | 60 | mg |
| | | Corticosteroid | | | | |
| | PLGA | Carrier/Release | 200 | mg | 200 | mg |
| | | controlling polymer | | | | |
| | Ethyl acetate | Solvent | 6 | mL | 6 | mL |
| | Acetone | Solvent | 4 | mL | 4 | mL |
| Aqueous | TMAB | Emulsion stabilizer/ | 26 | mg | 50 | mg |
| | | Surface modifier | | | | |
| | WFI | Solvent | 20 | mL | 20 | mL |

Both oil phase and aqueous phase solutions were filtered through 200 nm syringe filters prior to emulsification. The oil phase was emulsified in the aqueous phase by high-shear homogenization using a rotor/stator homogenizer operating at 16 k RPM. Immediately after homogenization started in TMAB solution, the organic phase was added dropwise with a syringe during 2 min. The resulting emulsion was homogenized for about 10 min. Solidification of PLGA nanoparticles, and thereby encapsulation of dexamethasone in nanoparticles, was triggered by transferring the O/W emulsion into a vessel containing 200 mL WFI under stirring. Stirring was continued for 4½ hours in a fume hood at room temperature (RT) in order to remove solvents. A milky suspension of nanoparticles was obtained. The suspension was further treated and particles were characterized through the same methods explained in Example 1. The characterization results are given in Table 5.

TABLE 5
Characteristics of dexamethasone-loaded positively-charged PLGA
nanoparticles

Formulation Lot #

| | | LBN0023- |
|---|---|---|
| Attributes | LBN0023-119-1 | 119-2 |
| Drug loading | 17% | 15% |
| Encapsulation Efficiency | 75% | 67% |
| Mean Particle Size | 186 nm | 169 nm |
| Polydispersity Index | 0.07 | 0.07 |
| Zeta potential | (+) 70 mV | (+) 64 mV |

It is evident that the drug loading of nanoparticles was smaller than that achieved with formulation LBN0023-076-1 of Example 1. The drug loading decreased when the cationic surfactant TMAB was used in the aqueous phase as emulsion stabilizer/surface modifier. Use of surfactant in the aqueous phase promotes partitioning of dexamethasone into this phase, reducing the nanoparticle drug loading.

### Example 3

### Fabrication of Methazolamide-Loaded Positively-Charged PLGA Nanoparticles

Three lots of methazolamide-loaded positively-charged PLGA nanoparticles were fabricated with water-in-oil emulsification-solvent diffusion-evaporation method. All three lots were prepared with PLGA 8515 DLG 1.5CE.

To prepare the oil phase, about 800 mg PLGA was dissolved in 20 mL ethyl acetate. About 5 mL of the PLGA solution was diluted with 2 mL ethyl acetate and 3 mL PEG400 (polyethylene glycol, MW 400). The solution was stirred for an additional hour to ensure complete dissolution of polymer. About 6 mg TMAB and about 200 mg methazolamide were added to the above solution and dissolved by stirring overnight. Aqueous phase consisted of a 1% (w/v) solution of PVA in WFI (10 mL). Target compositions of oil phase and aqueous phase solutions are provided in Table 6. Both oil phase and aqueous phase solutions were filtered through 200 nm syringe filters prior to emulsification. About 5 mL of the oil phase was emulsified in the aqueous phase by high-shear homogenization using a rotor/stator homogenizer operating at 16 k RPM. Immediately after homogenization started in PVA solution, the organic phase was added dropwise with a syringe during 30 seconds. The resulting emulsion was homogenized for about 5 min. Solidification of PLGA nanoparticles, and thereby encapsulation of methazolmide nanoparticles, was achieved by continuous stirring at 1200 RPM under atmospheric pressure at RT. Stirring was continued for about 4 hours in a fume hood in order to remove solvents. A milky suspension of nanoparticles was obtained. The suspension was transferred into centrifuge tubes, and then centrifuged at 6 k RPM for 15 minutes at 4° C. After the supernatant was removed, the pellet was washed with WFI (6 mL) by re-suspending the nanoparticles using a vortex mixer. The suspension was centrifuged again and the supernatant was removed. The pellet was reconstituted in WFI (6 mL) by vortexing and stored at 4° C.

TABLE 6
Target compositions of oil phase and aqueous phase solutions

| Emulsion | | | | |
|---|---|---|---|---|
| Phase | Ingredient | Function | Quantity | |
| Oil | Methazoalmide | Active agent/Antiglaucomatous | 200 | mg |
| | PLGA | Carrier/Release controlling | 100 | mg |
| | | polymer | | |
| | TMAB | Surface modifier/Surfactant | 3 | mg |
| | Ethyl acetate | Solvent | 3.5 | mL |
| | Acetone | Solvent | 1.5 | mL |
| Aqueous | PVA | Emulsion stabilizer/Surface | 100 | mg |
| | | modifier | | |
| | WFI | Solvent | 10 | mL |

Particle size and zeta potential measurements were performed by means of a Zetasizer Nano ZS90 (Malvern Instruments, Westborough, Mass.) using reconstituted nanoparticles in WFI.

About 1 mL of reconstituted suspension was transferred to a pre-weighed 1.5 mL eppendorf tube and centrifuged at 13.2 k RPM (RCF 16.1 k×g) for 25 minutes at RT. After the supernatant was removed, the pellet was dried under vacuum overnight at room temperature. The tube containing dry pellet was weighed and the weight of dry pellet was obtained. Dry pellet was then dissolved in 0.2 mL of dimethyl sulfoxide (DMSO). An aliquot of the resulting solution was analyzed by high performance liquid chromatography (HPLC).

Drug loading and encapsulation efficiency were calculated from Equations II and III, respectively.

The characterization results are given in Table 7.

TABLE 7
Characteristics of methazolamide-loaded positively-charged PLGA
nanoparticles

| | Formulation Lot # | | | |
|---|---|---|---|---|
| | LBN0032- | LBN0032- | LBN0032- | Ave. ± Std. |
| Attributes | 001 | 008-1 | 008-2 | Dev. |
| Drug loading | 45% | 53% | 50% | 49 ± 4 (%) |
| Encapsulation | 45% | 41% | 43% | 43 ± 2 (%) |
| Efficiency | | | | |
| Mean Particle Size | 290 nm | 288 nm | 289 nm | 289 ± 1 nm |
| Polydispersity Index | 0.07 | 0.04 | 0.01 | 0.04 ± 0.03 |
| Zeta potential | (+) 46 mV | (+) 47 mV | (+) 52 mV | (+) 48 ± 1 mV |

### Example 4

### In Vitro Release of Methazolamide from Methazolamide-Loaded Positively-Charged PLGA Nanoparticles in Phosphate Buffer (pH 7.4)

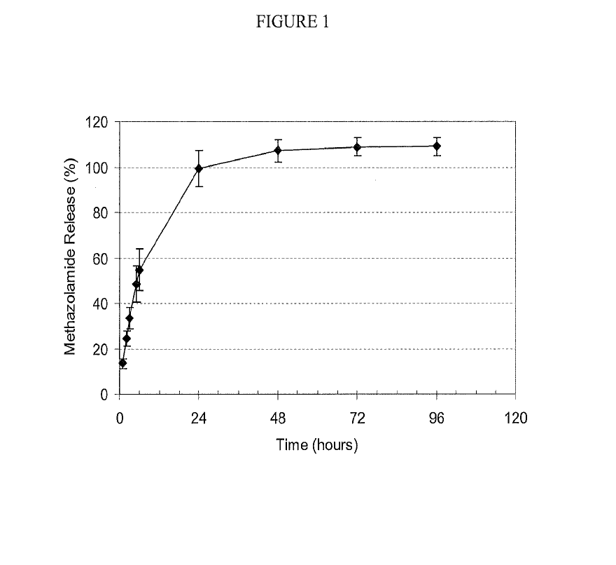

In vitro release of methazolamide from methazolamide-loaded positively-charged PLGA nanoparticles (lot # LBN0032-001, described in Example 2) was evaluated using 1-mL Float-A-Lyzer® dialysis bags, 3.5-5 kD MW cutoff (Rancho Dominguez, Calif.). Nanoparticles were dispersed in 1 mL of a phosphate buffer solution (pH 7.4, osmolality 293 mOsm/kg) in a dialysis bag. The bag, served as the donor chamber, was placed in a 50-mL Falcon™ tube, served as the receptor chamber and filled with about 20 mL of the same phosphate buffer solution. The release test was performed in quadruplicate. All release test tubes were placed in a shaking incubator at 37° C. with oscillation movement at 150 RPM. At predetermined time points the entire receptor solution was removed and replaced with a fresh solution. An aliquot of the collected receptor solution was analyzed for methazolamide concentration by HPLC. FIG. 1 shows the release profile of methazolamide-loaded PLGA nanoparticles.

### Example 5

### In Vivo Administration of an Active Agent Using a Composition of the Present Technology

This example is related to the treatment of a subject using a nanoparticle composition of this invention. The method of treatment is specifically related to anodal transscleral iontophoresis of a nanoparticle composition of the present technology by using a round shaped ocular device. The ocular device is equipped with a reservoir that can contain a given volume of nanoparticle suspension. Once the device is loaded with the nanoparticle suspension (typical concentration 0.1-10 mg/mL), it is placed on the anesthetized eye of the subject. The ocular device is designed to have the minimal surface of contact with the cornea if any. The ocular device is in electrical contact with a low-voltage generator which in turn is connected to a return (passive) electrode placed on a different point of the body surface of the subject. The nanoparticles are electro-mobilized once a low current typically in the range of about +1 mA to about +10 mA under a coulomb-controlled regimen is applied for a period of typically from about 1 min to about 10 min. The nanoparticles can be delivered to intraocular tissues by the process of electrorepulsion. With consideration of the route of delivery, such ocular tissues as conjunctiva, sclera, and iris/ciliary body initially receive a greater portion of the drug-loaded nanoparticles while a smaller portion of the nanoparticles is delivered to the choroid and the retina. Upon delivery the nanoparticles reside in the ocular tissues and sustain the release of their active contents as per their design.

### EQUIVALENTS

From the foregoing detailed description of the specific embodiments of the technology, it should be apparent that unique positively-charged poly(d, l-lactide-co-glycolide) nanoparticles capable of releasing a bioactive substance have been described as well as methods for their making and use. Although particular embodiments have been disclosed herein in detail, this has been done by way of example for purpose of illustration only, and is not intended to be limiting with respect to the scope of the appended claims which follow. In particular, it is contemplated by the inventors that various substitutions, alterations, and modifications may be made to the technology without departing from the spirit and scope of the technology as defined by the claims. For instance, the choice of QACS, or the route of administration is believed to be matter of routine for a person of ordinary skill in the art with knowledge of the embodiments described herein.

What is claimed is:

1. A nanoparticle composition comprising:
a) PLGA or a derivative thereof;
b) at least one quaternary ammonium cationic surfactant (QACS);
c) a permanent positive surface charge, represented by a positive zeta potential;
d) a particle size from at least about 10 nm to about 900 nm; and
e) at least one bioactive agent.

2. A composition of claim 1, wherein the zeta potential ranges from about +10 mV to about +100 mV.

3. A composition of claim 1 suitable for ocular administration.

4. A method of treating or preventing an ocular disease or condition in a subject, the method comprising administering to a subject in which such treatment or prevention is desired an amount of the composition of claim 1 in an amount sufficient to treat or prevent the ocular disease or condition in the subject.

5. The method of claim 4, wherein the ocular disease or condition is selected from the group consisting of: glaucoma, ocular inflammatory conditions such as keratitis, uveitis, intra-ocular inflammation, allergy and dry-eye syndrome ocular infections, ocular allergies, ocular infections (bacterial, fungal, and viral), cancerous growth, neo vessel growth originating from the cornea, retinal oedema, macular oedema, diabetic retinopathy, retinopathy of prematurity, degenerative diseases of the retina (macular degeneration, retinal dystrophies), and retinal diseases associated with glial proliferation.

6. A method for manufacturing the nanoparticle composition according to claim 1, comprising the steps of:
(a) preparing an oil phase by dissolving one or more bioactive agents, a one or more PLGA polymers, a one or more QACS, and optionally a one or more non-ionic surfactants in an organic solvent or a combination of organic solvents;
(b) preparing a water phase by dissolving one or more non-ionic polymeric stabilizers, optionally a one or more QACS, optionally a one or more non-ionic surfactants, and optionally a one or more pH modifying agents in purified water;
(c) emulsifying the oil and the water phase sonically, pneumatically, or mechanically under high-shear mixing;
(d) triggering the solvent diffusion-evaporation;
(e) solidifying the nanoparticles and encapsulating the active agent(s);
(f) separating the nanoparticles from the liquid medium by centrifugation or filtration; and
(g) removing the un-encapsulated ingredients from their surface by washing several times by purified water.

7. The method according to claim 6, wherein the organic solvent of step (a) has a normal boiling point from about 35° C. to about 85° C.

8. The method according to claim 6, wherein the step (d) is conducted by a method selected from the group consisting of: blending the emulsion with excessive amount of an aqueous solution; depressurizing the headspace of emulsion below the atmospheric pressure while mixing; maintaining the headspace of emulsion at the atmospheric pressure while mixing; heating the emulsion at a temperature between about 35 and about 45° C.; or any combination thereof.

9. A method for manufacturing the nanoparticle composition according to claim 1, comprising the steps of:
(a) preparing an internal water phase (dispersed phase of first emulsion) by dissolving a one or more bioactive agents, optionally a one or more QACS, optionally a one or more non-ionic surfactants, optionally a one or more non-ionic polymeric stabilizers, and optionally a one or more pH modifying agents in purified water;
(b) preparing an oil phase by dissolving a one or more PLGA polymers, a one or more QACS, optionally a one or more bioactive agents, and optionally a one or more non-ionic surfactants in an organic solvent or a combination of organic solvents;
(c) preparing an external water phase by dissolving a one or more non-ionic polymeric stabilizers, optionally a one or more QACS, optionally a one or more non-ionic surfactants, and optionally a one or more pH modifying agents in purified water;
(d) emulsifying the internal water phase with the oil phase sonically, pneumatically, or mechanically under high-shear mixing to form a first emulsion;
(e) emulsifying the first emulsion with the external water phase to form a double emulsion;
(f) triggering solvent diffusion-evaporation;
(g) solidifying the nanoparticles and encapsulating the active agent(s);
(h) separating the nanoparticles from the liquid medium by centrifugation or filtration; and
(i) removing un-encapsulated ingredients from the nanoparticle surface by washing several times by purified water.

10. The method according to claim 9, wherein step (d) is conducted by a method selected from the group consisting of: blending the emulsion with excessive amount of an aqueous solution; depressurizing the headspace of emulsion below the atmospheric pressure while mixing; maintaining the headspace of emulsion at the atmospheric pressure while mixing; heating the emulsion at mildly high temperatures, or any combination thereof.

11. The method according to claim 9, wherein the organic solvent of step (b) has a normal boiling point from about 35° C. to about 85° C.

12. A method for manufacturing the nanoparticle composition according to claim 1, comprising the steps of:
(a) preparing an at least two primary oil phases by dissolving a one or more bioactive agents, a one or more PLGA polymers, a one or more QACS, and optionally one or more non-ionic surfactants in respective organic solvent or organic solvent mixtures;
(b) preparing a water phase by dissolving a one or more non-ionic polymeric stabilizers, optionally a one or more QACS, optionally a one or more non-ionic surfactants, and optionally a one or more pH modifying agents in purified water;
(c) emulsifying the at least two primary oil phases in water phase concomitantly or in succession, sonically, pneumatically, or mechanically under high-shear mixing;
(d) triggering solvent diffusion-evaporation by any of the following methods:
(d.1) blending the emulsion with excessive amount of an aqueous solution;
(d.2) depressurizing the headspace of emulsion below the atmospheric pressure while mixing;
(d.3) maintaining the headspace of emulsion at the atmospheric pressure while mixing;
(d.4) heating the emulsion at a temperature between about 35° C. and about 45° C.;
(d.5) a combination of any of methods d.1 through d.3 with method d.4.;
(e) solidifying the nanoparticles and encapsulating the active agent(s);
(f) separating the nanoparticles from the liquid medium by centrifugation or filtration; and
(g) removing the un-encapsulated ingredients from their surface by washing several times by purified water.

13. The method for manufacturing the nanoparticle composition according to claim 12, wherein the organic solvent of step (a) has a normal boiling point from about 35° C. to about 85° C.

14. A method for manufacturing the nanoparticle composition according to claim 1, comprising the steps of:
(a) preparing an at least two internal water phases by dissolving a one or more bioactive agents, optionally a one or more QACS, optionally a one or more non-ionic surfactants, optionally a one or more non-ionic polymeric stabilizers, and optionally a one or more pH modifying agents in respective portions of purified water;
(b) preparing an at least two primary oil phases by dissolving a one or more PLGA polymers, a one or more QACS, optionally a one or more bioactive agents, and optionally a one or more non-ionic surfactants in respective organic solvent or organic solvent mixtures;
(c) preparing an external water phase by dissolving a one or more non-ionic polymeric stabilizers, optionally a one or more QACS, optionally a one or more non-ionic surfactants, and optionally a one or more pH modifying agents in purified water;
(d) emulsifying the at least two internal water phases, respectively, with the at least two oil phases sonically, pneumatically, or mechanically under high-shear mixing to establish an at least two first emulsions;
(e) emulsifying the at least two first emulsions with the external water phase, concomitantly or in succession, to form the double emulsion;
(f) triggering solvent diffusion-evaporation by any of the following methods:
(f.1) blending the emulsion with excessive amount of an aqueous solution;
(f.2) depressurizing the headspace of emulsion below the atmospheric pressure while mixing;
(f.3) maintaining the headspace of emulsion at the atmospheric pressure while mixing;
(f.4) heating the emulsion at a temperature between about 35° C. and about 45° C.;
(f.5) combination of any of methods f.1 through f.3 with method f.4;
(g) solidifying the nanoparticles and encapsulating the active agent(s);
(h) separating the nanoparticles from the liquid medium by centrifugation or filtration; and
(i) removing the un-encapsulated ingredients from their surface by washing several times by purified water.

15. The method for manufacturing the nanoparticle composition according to claim 14, wherein the organic solvent of step (b) has a normal boiling point from about 35° C. to about 85° C.
